(19) United States
(12) Patent Application Publication
(10) Pub. No.: US 2013/0078667 A1
(43) Pub. Date: Mar. 28, 2013

(54) METHODS FOR DETECTING AND COLLECTING CIRCULATING TUMOR CELLS

(71) Applicants: Oscar B. Goodman, Las Vegas, NV (US); Thuc Le, Las Vegas, NV (US)

(72) Inventors: Oscar B. Goodman, Las Vegas, NV (US); Wolfram Samlowski, JR., Las Vegas, NV (US); Thuc Le, Las Vegas, NV (US)

(21) Appl. No.: 13/240,824

(22) Filed: Sep. 22, 2011

(51) Int. Cl. G01N 21/75; C12N 5/09

(52) U.S. Cl. 435/34; 435/325; 435/395

(57) ABSTRACT

The present invention is directed towards methods of expanding circulating tumor cell populations and methods of detecting circulating tumor cells in a sample.

### BACKGROUND OF THE INVENTION

Early during tumor development, malignant cells gain the ability to enter the vasculature, circulate, adhere to endothelial cells, extravasate and grow in distant organs. Development of metastasis in vital organs remains the major cause of cancer-related mortality. CTCs are cells that have detached from a primary tumor and circulate in the bloodstream. CTCs provide the link between the primary and metastatic tumors. Detection and enumeration of CTCs in peripheral blood provides important prognostic information across a broad range of epithelial cancers, such as prostate, breast and colon cancer. The number of CTCs strongly correlates with cancer progression/regression and patient survival, and may antedate radiographic evidence of metastases. CTC assays also have predictive value that can guide treatment selection and protocols. Decreases in CTCs correlates with eventual tumor marker and radiologic response.

Thus the identification and characterization of CTCs could be useful for the early detection and treatment management of pre-metastatic and metastatic epithelial malignancies. For example, detection of CTCs in cancer patients could be an effective tool for early diagnosis of primary or secondary cancer growth and for predicting the prognosis of cancer patients undergoing cancer therapies because the number and characterization of CTCs present in the blood of cancer patients has been correlated with overall prognosis and response to therapy. The ability to accurately detect CTCs could also be an effective tool to monitor the course of treatment. Cristofanilli et al., N Med 351:781-791 (2004). CTCs can provide near real-time information about a patient's current disease state. However, because CTCs exist in such small numbers, it is difficult to obtain sufficient amounts of CTCs for characterization using a standard blood draw.

Previous devices and methods for collecting CTCs have limitations. All previous techniques rely on a selection criteria, such as size, cell surface antigen, etc., which restrict the identified cell population. Another limitation in these devices and methods is that the sample volume is limited, and, therefore, the number of CTCs collected is small and limited. In many methods, the sample collection and preservation uses a fixative that devitalizes the cells. In some methods, the CTCs are captured using beads that impart further mechanical damage to the collected cells. Thus, in these systems, analysis of the collected CTCs is mainly limited to quantification due to the deleterious effects on the collected cells. As these systems detect relatively low numbers of cells, they are therefore limited in the types of diseases that can be identified, diagnosed or staged. For example, these systems detect cells only in patients with the highest disease burdens, such as metastatic patients.

Current assays are capable of identifying CTCs and correlating them with disease, however none of the current methods have sufficient sensitivity to reliably measure a statistically significant number of cells at varying stages of the disease. An immunomagnetic expansion method that has been developed relies on monoclonal antibodies conjugated to small magnetic beads that target the epithelial cell adhesion molecule, EpCAM. The beads are manipulated in magnetic fields for expansion of the CTCs. However, expression levels of EpCAM in CTCs are known to be substantially reduced from the levels of cells in tissues. Rao et al., Int J Oncol 27:49-57 (2005). Since sensitivity loss was observed in expansion of cells with reduced EpCAM expression, this approach would have low sensitivity for some CTCs. Krivacic at al., Proc Natl Acad Sci USA 101:10501-10504 (2004).

Another method for enumeration/characterization of CTCs is Fiber-optic Array Scanning Technology (FAST). Using the FAST method, 7.5 mL of blood is used for analysis. Red blood cells are lysed and nucleated cells are distributed as a monolayer on slides that can accommodate up to 30 million cells. There is no expansion step in this methodology. Cells are fixed, permeabilized and stained with a pan anti-cytokeratin antibody—Alexa Fluor 555, CD45-Alex Fluor 647 and DAPI (nuclear stain). FAST scans each slide and identifies the location of each red fluorescent object on the slide. Each fluorescent object is imaged via an automated digital microscope and CTCs are enumerated as being CK+, CD45−, DAPI+ cells. This methodology has been tested on a variety of metastatic cancer patients, including breast, lung, prostate, colorectal, and pancreatic. Similar CTC counts are found using this method as compared to methods using immunomagnctic expansion. Hsich, ct al., Biosensors and Bioelectronics, 21: 1893-1899 (2006).

Thus there is a need for methods to detect CTCs with sufficient sensitivity and selectivity to provide an accurate representation (count) of the amount of CTCs in a patient. Further, there is a need to detect and collect CTCs in a manner that does not alter the cells' characteristics and preserves its native form and function.

### SUMMARY OF THE INVENTION

The invention relates to methods of expanding a circulating tumor cell population, comprising providing a cell population containing circulating tumor cells (CTCs) and culturing the cell population under conditions suitable for preferential expansion of the CTCs. In some embodiments the cell population containing CTCs is obtained from a subject. The cell population containing CTCs may be obtained from any suitable biological sample, such as a blood sample, bone marrow, metastasis, or fresh prostate biopsy specimens. Preferably, the cell population containing CTCs is obtained from a peripheral blood sample. In some embodiments, the method further comprises obtaining a sample that contains cell population containing CTC from a subject. The sample may be a blood sample. The method may further comprise substantially removing red blood cells from the blood sample. The red blood cells may be removed, for example, using fractionation, red blood cell lysis, cell sorting, filtration, adhesion, density centrifugation, or a combination of methods. The cell population containing CTCs may be obtained from a cell culture.

The cell population containing CTCs can be cultured in a three dimensional matrix or spheroid culture, for example. The matrix may be selected from the group consisting of methylcellulose, collagen and matrigel. The matrix may be solid or semisolid. The depth of the matrix can be 0.1-1 cm thick. The cell population containing CTCs can be cultured in media that contains less than about 5% serum or plasma. The serum or plasma may be human scrum. The cell population containing CTCs is cultured for at least about one week. At the end of the culture period, the number of CTCs can be at least about one hundred times (100×) the number of CTCs at the start of the culture period. The ratio of CTCs to leukocytes at the end of the culture period is at least about 100:1, corresponding to an enrichment factor of at least 10⁹. The method may further comprise incorporating non cancer-derived extrinsic cells into the culture.

The invention also relates to methods of detecting CTCs in a sample, comprising providing a cell sample and detecting lipid-rich structures in the cells contained in the sample, wherein the presence of lipid-rich structure in a cell indicates that the cell is a CTC. The detecting can be done using a cellular lipid stain. The cellular lipid stain can be selected from the group consisting of Nile Red, Oil Red O, Dil, DiO, DiA, DiD and DiR. The sample may be further analyzed using CARS or RAMAN.

The invention also relates to a method of detecting CTCs in a sample, comprising staining a cell sample with a reagent that comprises a lipid stain under conditions wherein leukocytes are not stained, but CTCs are stained, and detecting stained cells. The reagent can comprise a lipid stain at a concentration of 100 nM. The reagent can further comprise a nuclear stain. The nuclear stain can be DAM or Hoeschst 33342. Preferably, the reagent comprises DAPI at a concentration of 20 nM.

### BRIEF DESCRIPTION OF THE DRAWINGS

FIG. 1 shows the preferential expansion of CTCs in cultures of peripheral blood cells in methyl cellulose based culture media. The colonies represent the growth of CTCs at day 8 (FIG. 1A) and day 10 (FIG. 1B). Peripheral blood cells that were cultures were isolated from the peripheral blood of a prostate cancer patient. The bar represents 200 μm.

FIG. 2A-L micrographs of cultures of peripheral blood cells from patients with prostate cancer showing expansion of CTCs, Patient 1 (A-C), Patient 2 (D-F), Patient 3 (G-I) and Patient 4 (J-L), in spheroid cultures. Only cells obtained from Patient 1 grew in the spheroid culture (A-C).

FIGS. 3A-D are micrographs showing the time-course of CTCs expansion from Patient 1 in spheroid culture.

FIG. 4A-L are micrographs of cultures of peripheral blood cells from patients with prostate cancer showing expansion of CTCs, Patient 1 (A-C), Patient 2 (D-F), Patient 3 (G-I) and Patient 4 (J-L), in three dimensional methyl cellulose culture. Cells obtained from all four patients grew in the three-dimensional culture.

FIGS. 5A-D are micrographs showing the time-course of CTCs expansion from Patient 1 in three-dimensional culture.

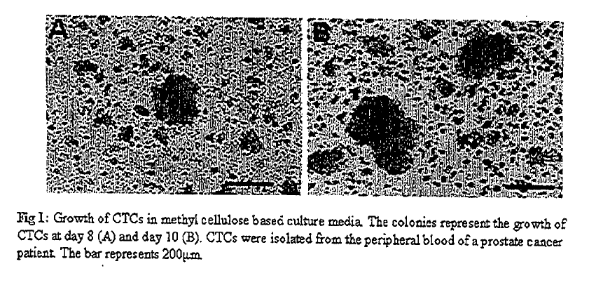

### DETAILED DESCRIPTION

The present invention relates generally to cancer diagnostics, prognostics, monitoring and therapy, and more specifically to methods for detecting and expanding circulating tumor cells in a sample (e.g, a patient sample, blood etc.), to make them more easily detectable and collectible as well as more readily available for meaningful characterization, thereby allowing for clinical analysis and diagnostic and therapeutic applications. The CTCs can also be used in research into novel tumor markets, novel drug targets, understanding molecular pathways and cancer biology as well as the treatment, diagnosis and prognosis of cancer, or to develop therapeutics (e.g., antibodies).

The detection of circulating tumor cells (CTCs) allows for near real-time information about a patient's current disease state. The methods of this invention provide the ability to detect a clinically sufficient number of CTCs in a single procedure, providing a positive impact on improving the overall care of cancer patients. The methods described herein include screening, prognostics, diagnostics, monitoring and therapeutics.

These methods result in approximately 10,000 fold increase in the sensitivity of CTC detection. This is accomplished by expanding the CTC population using the ex vivo method described herein. CTCs can be collected using any suitable method, such as through traditional, known blood sampling techniques, and then selectively expanded, thus making them more abundant and therefore, more easily detected and studied.

Another advantage of the methods described herein is that viable CTCs can be derived from a variety of biological sources (e.g., whole blood) and detected independently of selection criteria (i.e., antibody binding affinity for lipid uptake) and/or grown independently (e.g., favoring those CTCs with replicative potential).

Diseases or other medical conditions for which the invention described herein are applicable include, but are not limited to, any of a variety of cancers or other neoplastic conditions. This includes, for example, epithelial cell cancers such as lung, ovarian, cervical, endometrial, breast, brain, colon and prostate cancers. Also included are gastrointestinal cancer, head and neck cancer, non-small cell lung cancer, cancer of the nervous system, kidney cancer, retina cancer, skin cancer, liver cancer, pancreatic cancer, genital-urinary cancer, bladder cancer, melanoma and leukemia. In addition, the methods and compositions of the present invention are equally applicable to detection, diagnosis and prognosis of non-malignant tumors in an individual (e.g., neurofibromas, meningiomas, and schwannomas).

### DEFINITIONS

CTCs may be expanded in any suitable sample type. As used herein, the term “sample” refers to any sample suitable for the methods provided by the present invention. The sample may be any sample that includes CTCs. Sources of samples include whole blood, bone marrow, pleural fluid, peritoneal fluid, central spinal fluid, metastasis, fresh biopsy samples (e.g., fresh prostate biopsy sample), urine, saliva and bronchial washes. In one aspect, the sample is a blood sample, including, for example, whole blood or any fraction or component thereof. A blood sample, suitable for use with the present invention may be extracted from any source known that includes blood cells or components thereof, such as veinous blood, arterial blood, peripheral blood, tissue, cord blood, and the like. For example, a sample may be obtained and processed using well known and routine clinical methods (e.g., procedures for drawing and processing whole blood). In one aspect, a sample may be peripheral blood drawn from a subject with cancer.

The term “circulating tumor cell” (CTC) is intended to mean any circulating cancer cell that is found in a sample obtained from a subject. Typically, CTCs have been shed from a solid tumor. As such, CTCs are often epithelial cells shed from solid tumors that are found in very low concentrations in the circulation of patients with advanced cancers. CTCs may also be mesothelial cells from sarcomas or melanocytes from melanomas.

The term “early stage cancer” as used herein refers to those cancers which have been clinically determined to be organ-confined. Also included are tumors too small to be detected by conventional methods such as mammography for breast cancer patients, or X-rays for lung cancer patients. While mammography can detect tumors having approximately 2×10⁸ cells, the methods of the present invention should enable detection of CTCs from tumors approximately this size or smaller.

The term “expansion” as used herein refers to an increase in the number of CTCs, e.g., in a biological sample or culture. Accordingly, a cell population or an expanded CTC population is intended to mean a sample that has been cultured as described herein to increase the number of CTCs as compared to if the sample had not been cultured. For example, the number of CTCs in a sample may be increased by at least about 10%, 25%, 50%, 75%, 100% or by a factor of at least 2, 5, 10, 20, 30, 40, 50, 60, 70, 80, 90, 100, or 200. Using the method of the present invention, CTCs may be expanded relative to leukocytes to the extent of at least about 2,500 fold, at least about 5,000 fold, or at least about 10,000 fold.

The term “subject” as used herein refers to any individual or patient from whom CTCs (or a sample containing CTCs) is obtained or to whom the subject methods are performed. Generally the subject is human, although the subject may be an animal, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas).

### Methods

### A. Expansion of CTCs in Cell Culture

In one aspect, the invention relates to methods for expanding a CTC population. Accordingly, in one embodiment the method comprises providing a cell population containing CTCs and culturing the cell population under conditions suitable for preferential expansion of CTCs.

The cell population containing CTCs may be obtained from any suitable source such as from a sample taken or obtained from a subject, from an existing CTC culture, from a frozen cell stock, and the like. In preferred embodiments, the cell population containing CTC is obtained in a sample taken from a subject. Suitable samples include, for example, blood (e.g., whole blood), bone marrow (disseminated tumor cells), metastasis, fresh biopsy (e.g., fresh prostate biopsy specimen) samples and the like. In a particular embodiment, the sample is a blood sample, such as a peripheral blood sample.

When CTCs are present in a sample obtained from a subject, the sample can be processed to remove or reduce the number of non-CTC in the sample (i.e., to enrich for CTC) and the resulting cell population that contains CTC can be cultured in accordance with the methods described herein. Alternatively, the sample can be cultured without further processing.

In preferred embodiments, a blood sample obtained from a subject is processed to enrich for CTC prior to culturing. For example, a peripheral blood sample can be processed to substantially remove red blood cells and/or other types of blood cells from the blood sample using any suitable methods. Many suitable methods for removing red blood cells and/or other types of blood cells from whole blood are conventional and well-known in the art (e.g., fractionation, red blood cell lysis, cell sorting, filtration, adhesion, density centrifugation, ammonium chloride lysis). For example, red blood cells can be removed from a whole blood sample by density gradient sedimentation. Typically, the process relies on a gross physical distinction, such as cellular density for separating nucleated cells such as CTCs from erythrocytes and, optionally, other non-CTC cells. Many variations of this general method are well known in the art and suitable for use in the methods of the present invention. For example, whole blood samples can be anticoagulated using heparin or EDTA and cells can be fractionated by centrifugation through Ficoll-Hypaque and/or red blood cells can be lysed using ammonium chloride lysis. Many other suitable methods to isolate circulating nucleated cells from red blood cells and other components of blood are well known in the art.

In one embodiment, a blood sample is provided and blood cells are substantially removed from the blood sample.

The cell population containing CTCs can be cultured in any suitable type of culture, such as a suspension culture, spheroid culture or cultured in a matrix. Preferably, the cell population containing CTCs is cultured in a matrix, which can be a solid or semi-solid matrix. Any coated or uncoated vessel, flask, or three-dimensional extracellular matrix that facilitates cell attachment, growth, differentiation, migration, and tissue morphogenesis can be used. For example, the matrix may be methylcellulose, carboxymethylcellulose, collagen, Matrigel™ (basement membrane preparation extracted from the Enelbreth-Holm-Swam mouse sarcoma; BD Biosciences), and the like. The depth of the matrix may be between 0.0-1 cm, 0.0-2 cm, or 0.0-3 cm thick.

In a particular embodiment, the method further comprises obtaining a sample that contains a cell population containing CTCs from a subject. The sample may be a peripheral blood sample from a subject. In one aspect, the method comprises isolating the buffy coat from the subject's peripheral blood, plating recovered cells (i.e., white cells and tumor cells) into media, and culturing the cells under conditions suitable for preferential expansion of CTC (e.g., 5% CO₂, 37° C. incubator). Cells may be cultured for about 1 week, about 2 weeks, about 3 weeks, about 4 weeks, about 1-2 weeks, or about 2-4 weeks.

The culture media can be any media suitable for growth of mammalian (e.g., human) cells, and typically contain salts, amino acids and other nutrients, and can be supplemented with antibiotics, and other components if desired. Many suitable media formulations are well known and conventional in the art, such as RPMI media, Knockout serum replacement media, F12K, and DMEM. The culture media is preferably supplemented with a low concentration of serum, plasma or growth factors (i.e. 5% or less (v/v) serum or plasma) or is serum or plasma free. These conditions favor the growth of tumor cells under epithelial to mesenchymal transition (or “stem cell-like”) conditions. For example, the white blood cells from the buffy coat will not proliferate without addition of specific growth factors and higher serum concentration, but as shown herein CTCs will proliferate under these conditions.

The culture media may comprise a matrix (e.g., cell culture media with less than 5% human serum and 0.1%-10%, preferably 0.1%-1.6% Matrigel or other suitable matrix). The matrix-based media may comprise about 0.1% to about 10%, about 0.1% to about 5%, about 0.1% to about 1.6%, 0.1% to about 1%, at least about 0.5% matrix, at least about 0.6% matrix, at least about 0.6% matrix, at least about 0.7% matrix at least about 0.8% matrix, at least about 0.9% matrix, at least about 1.0% matrix, at least about 1.2% matrix, at least about 1.4% matrix, at least about 1.5% matrix, at least about 1.6% matrix, at least about 1.7% matrix, at least about 1.8% matrix, at least about 2.0% matrix, at least about 2.4% matrix, at least about 2.6% matrix, at least about 2.8% matrix, at least about 3.0% matrix, or at least about 3.2% matrix. The matrix-based media may comprise less than 5% serum or plasma (e.g., human serum or plasma).

The cell culture media may comprise about 0.1% to about 5%, about 0.1% to about 4%, about 0.1% to about 3% scrum or plasma (e.g., human scrum or plasma), about 0.5% to about 2.5% serum or plasma (e.g., human serum or plasma), about 1% to about 2% serum or plasma (e.g., human serum or plasma), about 0.5%, about 0.6%, about 0.7%, about 0.8%, about 0.9%, about 1.0% about 1.2%, about 1.4%, about 1.6%, about 1.8%, about 2.0%, about 2.2%, about 2.4%, about 2.6%, about 2.8% or about 3% serum or plasma (e.g., human serum or plasma). The culture media preferably is supplemented with a small amount (i.e., 5% (v/v) or less) of human serum or plasma or is serum free.

In a particular embodiment, the matrix-based media may contain about 0.1% to about 1.6% methyl cellulose, RPMI medium (1× or 2×) and about 2% human serum.

In some embodiments, non-cancer derived extrinsic cells are incorporated into the culture to improve the growth of the culture. Suitable extrinsic cells include hematopoetic mesenchymal stem cells and leukocytes, such as monocytes, macrophages and lymphocytes.

Suitable culture conditions may comprise incubation at about 35° C. to about 40° C. (e.g., about 36° C., about 37° C., about 38° C., about 39° C.) in a humidified atmosphere (e.g. 95% relative humidity) that contains about 3% to about 7% carbon dioxide (e.g., about 5% carbon dioxide). Suitable culture conditions also comprise incubation for a period of time sufficient for expansion of CTCs, such as, for example, about 3 to about 21 days, e.g., about 5 days, about 6 days, about 7 days, about 8 days, about 9 days, about 10 days, about 14 days, or about 20 days.

The number of CTCs at the end of the culture period may be at least 50×, at least 100×, at least 150×, or at least 200× the number of CTCs present at the start of the culture period.

The ratio of CTCs to leukocytes at the end of the culture period may be at least 50:1, at least 75:1, at least 100:1, at least 125:1, at least 150:1, at least 175:1, or at least 200:1. Preferably the ratio is at least 100:1, corresponding to an expansion factor of at least 10⁷.

CTC colonies that form in the cultures can be identified in any suitable way. For example, CTC colonies may be evaluated by inverted microscopy, and may be recovered for further characterization. The cultures may be used to identify and score in a quantitative or semi-quantitative way the presence of CTCs (e.g., metastatic melanoma, prostate cancer cells) in the circulation of subjects for use as a prognostic and/or predictive marker.

In various embodiments of the present invention, expanded CTCs are analyzed to derive clinically significant data. Analysis of CTCs may be performed by a variety of methods depending on the type of data desired. For example, in various aspects, subsequent to expanding the CTCs, CTCs may be analyzed by detecting and characterizing the CTCs via assays utilizing recognition and/or binding of cellular components, such as cell surface markers. A variety of detection/immobilization assays can be used with the present invention from which useful data may be derived. Additional analysis methods may include image analysis.

As used herein, image analysis includes any method which allows direct or indirect visualization of expanded CTCs. For example, image analysis may include, but is not limited to, ex vivo microscopic or cytometric detection and visualization of cells bound to a solid substrate, flow cytometry, fluorescent imaging, and the like. In an exemplary aspect, expanded CTCs are detected using antibodies directed to cell surface markers and subsequently bound to a solid substrate and visualized using microscopic or cytometric detection. Additionally, the CTCs may be expanded ex vivo and reinfused into the subject and subsequently analyzed via imaging analysis.

In various embodiments, a variety of cell markers (e.g., surface markers) may be used to analyze and detect expanded CTCs. As used herein, cell markers include any cellular component that may be detected within or on the surface of a cell, or a macromolecule bound or aggregated to the surface of the cell. As such, cell markers are not limited to markers physically on the surface of a cell. For example, cell markers may include, but are not limited to surface antigens, transmembrane receptors or coreceptors, macromolecules bound to the surface, such as bound or aggregated proteins or carbohydrates, internal cellular components, and the like. In one aspect, the cell marker may be a cell adhesion molecule, such as EpCAM or a cytokeratin. For example, the antibodies used to detect cell markers may be anti-cytokeratin, pan-kerartin and anti-EpCAM.

Additionally, a number of cell markers known to be specific to cancers may be targeted or otherwise utilized to detect and analyze CTCs. For example, various receptors have been found to be expressed or over expressed only in particular types of cancers. In various aspects of the invention cell markers include EGFR, HER2, ERCCl, CXCR4, EpCAM, E-Cadherin, Mucin-1, Cytokeratin, PSA, PSMA, RRM1, Androgen Receptor (AR), Estrogen Receptor, Progesterone Receptor, IGF1, cMET, EML4, or Leukocyte Associated Receptor (LAR). Further, cell markers may be utilized that are specific to particular cell types. For example, useful endothelial cell surface markers include CD105, CD106, CD144, and CD146, while useful tumor endothelial cell surface markers include TEM1, TEM5, and TEM8.

B. Detection of CTCs based on Intracellular Lipid Content

In another aspect, the invention relates to methods of detecting CTCs based on intracellular lipid content. Cancer cells (e.g., metastatic melanoma and prostate cancer cells) differ markedly from circulating leukocytes based on their intracellular lipid content. For example, CTCs contain lipid-rich structures, which are intracellular lipid droplets, but leukocytes generally do not. Furthermore, red blood cells, erythrocytes, platelets, and thrombocytes do not have intracellular lipid droplets. Thus, CTCs can be clearly detected from the blood based on intracellular lipid content. The ability to detect and characterize CTCs has the potential to aide in the diagnostic and Individualized treatment of cancer subjects (e.g., personalized medicine).

In one embodiment, the method generally comprises providing a cell sample and detecting lipid-rich structures in the cells contained in the sample, wherein the presence of lipid-rich structures in a cell indicates that the cell is a CTC. The cell sample can be any of the cell populations that contain CTCs that are obtained from a subject us described herein, with or without processing. Preferably the cell sample comprises CTCs that are expanded, for example using the culture method described herein. Detecting the lipid-rich structures may be done using a reagent that contains a suitable cellular lipid stain. Suitable cellular lipid stains include Nile Red (e.g., Nile Red dye mixed with DAPI), Oil Red O, and the Di I family of fluorescent probes (e.g., Dil, Di O, DiA, DiD, DiR Molecular Probes). This method provides specific identification of circulating tumor cells based on a unique physical property, their lipid content.

In another embodiment, the method comprises staining a cell sample with a reagent that comprises a lipid stain under conditions wherein leukocytes are not stained, but CTCs are stained, and detecting stained cells. A concentration of cellular lipid stain that does not stain leukocytes, but allows for their visualization via nuclear staining may used. Appropriate concentrations include about 50 nM, about 60 nM, about 70 nM, about 80 nM, about 90 nM, about 100 nM, about 120 nM, or about 140 nM lipid stain. The cellular lipid stain may be diluted in Hanks buffered Salt Solution, or any suitable diluent.

The reagent may further comprise a nuclear stain. Suitable nuclear stains include DAPI, Hoeschst 33342, and the like. Appropriate concentrations of nuclear stain include about 10 nM, about 20 nM, about 30 nM, about 40 nM, or about 50 nM nuclear stain. In a preferred embodiment, the reagent comprises about 100 nM of Nile Red lipid stain and about 20 nM of DAPI nuclear stain. Using this type of stain, CTCs will stain with both the lipid stain and the nuclear stain, but leukocytes will only stain with the nuclear stain.

Presence of the circulating tumor cells in the sample indicates presence of cancer (e.g., an early stage cancer, metastatic cancer, etc.) in the subject. The absence of the circulating tumor cells in the sample indicates a cancer free state in the subject. In a further aspect, changes in the number of circulating tumor cells detected using the methods described herein during the course of therapy (e.g., at initiation of therapy, at regular intervals during therapy) can be used to monitor cancer therapy or cancer recovery. For example, a decrease in the number of CTCs detected during the course of therapy indicates the therapy is effective in treating the cancer. Alternatively, for example, an increase in the number of CTCs detected during the course of therapy indicates the therapy is not effective in treating the cancer, and an alternate therapy should be pursued.

### Verification of Detection

Known techniques, for example CARS microscopy and RAMAN micro-spectroscopy, may be used to verify the identity of CTCs isolated and detected by the methods of the invention. CARS imaging is label-independent, which allows for screening of thick smears of subject-derived buffy coat material and identification of potential target cells with fluorescent lipid stains, for efficient second step validation of CTCs. Raman micro-spectroscopy for lipid composition analysis may also be used. A spectrometer is added to the CARS microscope for spontaneous Raman micro-spectroscopy analysis. White blood cells and CTCs have differing Raman lipid spectroscopic signatures, allowing for further confirmation of their respective identities.

CARS signal of CTCs arises mostly from lipid droplets; whereas, CARS signal of leukocytes arises mostly from cell membrane. Correspondingly, RAMAN signature of CTCs resemble that of triglyceride, which is the major component of lipid droplets; whereas, RAMAN signature of leukocytes resembles that of phospholipid, which is a major component of cell membrane. Generally, CTCs exhibit 7-fold higher CARS signal intensity for CTCs than leukocytes. The differences in CARS signal intensity and RAMAN signature can be used to differentiate CTCs from leukocytes.

Alternatively, lipids that contain a stable isotope of hydrogen, such as deuterium, can be added to blood samples. CTCs will uptake deuterated lipids rapidly and incorporate them into intracellular lipid droplets. Using this approach, CTCs can further be discriminated from leukocytes based on C-D stretch vibration of deuterated lipid at 2100 cm⁻¹. Leukocytes lack the ability to uptake lipid and to accumulate intracellular lipid droplet. Therefore, RAMAN signature of leukocytes arises only from C—H stretch vibration at 2850 cm⁻¹.

### C. Characterization and Analysis of CTCs

Several known methods for counting, analyzing and characterizing the CTCs expanded and detected using the methods described herein can be used. Expansion, detection, and characterization of CTCs, using the methods of the invention, is useful in assessing cancer prognosis and in monitoring therapeutic efficacy for early detection of treatment failure that may lead to disease relapse. In addition, CTC analysis according to the invention enables the detection of early relapse in pre-symptomatic subjects who have completed a course of therapy. This is possible because the presence of CTCs has been associated and/or correlated with tumor progression and spread, poor response to therapy, relapse of disease, and/or decreased survival over a period of time. Thus, enumeration and characterization of revealed CTCs provides methods to stratify subjects for baseline characteristics that predict initial risk and subsequent risk based upon response to therapy.

Accordingly, in another embodiment, the invention provides a method for diagnosing or prognosing cancer in a subject. The method includes expanding circulating tumor cells of the subject as described herein. Expanded CTCs may then be analyzed to diagnose or prognose cancer in the subject. As such, the methods of the present invention may be used, for example, to evaluate cancer subjects and those at risk for cancer, e.g., early detection. In any of the methods of diagnosis or prognosis described herein, either the presence or the absence of one or more indicators of cancer, such as, a cancer cell, or of any other disorder, may be used to generate a diagnosis or prognosis.

In one aspect, a blood sample is drawn from the subject and CTCs in the sample are expanded as described herein. Using the method of the invention, the number of CTCs in the expanded culture is determined and the CTCs may be subsequently analyzed. For example, the cells may be labeled with one or more antibodies that bind to a cell adhesion molecule or cytokeratin, such as EpCAM, pan-keratin, anti-cytokeratin, or a tumor antigen such as anti-PSA, and the antibodies may have a covalently bound fluorescent label. Analysis may then be performed to determine the number and characterization of CTCs in the expanded sample, and from this measurement, the number of CTCs present in the initial blood sample may be determined. The number of CTCs may be determined by cytometric or microscopic techniques to visually quantify and characterize the CTCs.

In various aspects, analysis of a subject's CTC number and characterization may be made over a particular time course in various intervals to assess a subject's progression and pathology. For example, analysis may be performed at regular intervals such as one day, two days, three days, one week, two weeks, one month, two months, three months, six months, or one year, in order to track level and characterization of CTCs as a function of time. In the case of existing cancer subjects, this provides a useful indication of the progression of the disease and assists medical practitioners in making appropriate therapeutic choices based on the increase, decrease, or lack of change in CTCs, such as the presence of CTCs in the subject's bloodstream. Any increase (e.g., 2-fold, 5-fold, 10-fold or higher), in the expanded CTCs obtained from subject samples over time decreases the subject's prognosis and is an early indicator that the subject should change therapy. Similarly, any increase (e.g., 2-fold, 5-fold, 10-fold or higher), indicates that a subject should undergo further testing such as imaging to further assess prognosis and response to therapy. Any decrease (e.g., 2-fold, 5-fold, 10-fold or higher), in the CTCs over time shows disease stabilization and a subject's response to therapy, and is an indicator to not change therapy. For those at risk of cancer, a sudden increase in the number of CTCs detected may provide an early warning that the subject has developed a tumor thus providing an early diagnosis. In one embodiment, the detection of CTCs increases the staging of the cancer.

In any of the methods provided herein, additional analysis may also be performed to characterize CTCs, to provide additional clinical assessment. For example, in addition to image analysis and bulk number measurements, PCR techniques may be employed, such as multiplexing with primers specific for particular cancer markers to obtain information such as the type of tumor, from which the CTCs originated, metastatic state, and degree of malignancy. Additionally, cell size, DNA or RNA analysis, proteome analysis, or metabolome analysis may be performed as a means of assessing additional information regarding characterization of the subject's cancer. In various aspects, analysis includes antibodies directed to or PCR multiplexing using primers specific for one or more of the following markers: EGFR, 1-IER2, ERCC1, CXCR4, EpCAM, E-Cadherin, Mucin-1, Cytokeratin, PSA, PSMA, RRM1, Androgen Receptor, Estrogen Receptor, Progesterone Receptor, IGF1, cMET, EML4, or Leukocyte Associated Receptor (LAR).

For example, the additional analysis may provide data sufficient to make determinations of responsiveness of a subject to a particular therapeutic regime, or for determining the effectiveness of a candidate agent in the treatment of cancer. Accordingly, the present invention provides a method of determining responsiveness of a subject to a particular therapeutic regime or determining the effectiveness of a candidate agent in the treatment of cancer by expansion of CTCs of the subject as described herein and analyzing the expanded CTCs. For example, once a drug treatment is administered to a subject, it is possible to determine the efficacy of the drug treatment using the methods of the invention. For example, a sample taken from the subject before the drug treatment, as well as one or more cellular samples taken from the subject concurrently with or subsequent to the drug treatment, may be processed using the methods of the invention. By comparing the results of the analysis of each processed sample, one may determine the efficacy of the drug treatment or the responsiveness of the subject to the agent. In this manner, early identification may be made of failed compounds or early validation may be made of promising compounds.

### Additional Applications

Additional applications for using the isolated and expanded CTCs include, immunophenotyping, gene and protein expression profiling, micro RNA expression profiling, drug sensitivity testing, vaccine generation, monoclonal antibody generation, and treatment response assessment. The isolation of CTCs can be accomplished, for example, by using antibodies, aptamers, aptamer analogs or molecularly imprinted polymers specific for a desired surface antigen. By employing appropriate monoclonal antibodies directed to associated markers on or in target cells, or by using other assays for cell protein expression, or by the analysis of cellular mRNA, the organ origin of CTCs may readily be determined, e.g., breast, prostate, colon, lung, ovarian or other non-hematopoietic cancers.

In one aspect, an expanded CTC population may be administered to a subject alone to provide a therapeutic and/or prophylactic cancer vaccine. The expanded CTCs may be derived from the same subject they are isolated from or from a different subject. In another aspect, the expanded CTC population may be coadministered with a therapeutic agent, such as a targeted drug or chemotherapeutic drug. Virtually any known therapeutic drug or chemotherapeutic agent may be coadministered with the expanded CTCs.

The content of each of the patents, patent applications, patent publications and published articles cited in this specification are herein incorporated by reference in their entirety.

The following examples are provided to further illustrate the embodiments of the present invention, but are not intended to limit the scope of the invention. While they are typical of those that might be used, other procedures, methodologies, or techniques known to those skilled in the art may alternatively be used.

### EXEMPLIFICATION

### Example 1

### Isolation and Culturing of Prostate Cancer Circulating Tumor Cells (CTCs)

Materials: Ficoll-paque premium (density 1.077 g/ml) was purchased from GE Healthcare, (SWEDEN), and Methocel E4M premium was from DOW Chemical company, (Michigan, USA). RPMI, DMEM/F12, knock out serum were from Life Technologies (Grand Island, N.Y.). Human plasma used was from an unknown donor, non-essential amino acid was from Fisher Scientific (Fair Lawn, N.J.) and 2-beta mercaptoethanol (BME) was from Sigma Aldrich (St. Louis, Mo.) and human EGF was from R&D Systems (Minneapolis, Minn.).

Cell lines: The prostate cancer cell line LNCaP was from ATCC (Manassas, Va.). The prostate cancer cell line C4-2 was a gift from Dr. David Nanus (Cornell University) and melanoma cell line M14 was a gift from Dr. Wolfram Samlowski (Comprehensive Cancer Center, Las Vegas, Nev.) and were all grown in RPMI media.

Blood sample collection: The peripheral blood from the metastatic prostate cancer patients was collected according to an IRB approved protocol in heparin containing tubes. Blood samples were collected from four prostate cancer patients. Healthy volunteer blood was collected for isolation of leukocytes.

Preparation of methocel solution: Methocel solution was used for 3-dimentional culturing of CTCs. A 1.6% stock solution was prepared for this purpose. To prepare this stock 1.6 gm of methocel was resuspended in 30 ml of hot water (80-90° C., tissue culture grade) for 20 minutes using a stirrer; slowly 70 ml of cold water (4° C.) was poured with continuous mixing for another 20 minutes. After a clear solution was obtained, the stock solution was autoclaved, autoclaving makes the solution turbid and the methocel precipitates. The hot stock solution was again stirred continuously till it became transparent and cooled to room temperature. The stock was stored at room temperature until used.

Separation of Buffy coat: Ficoll reagent (3.5 ml) was poured into a transparent scaled 15 ml cell culture tube, the peripheral blood (7.5 ml) was layered slowly on top of the ficoll reagent. The tube was centrifuged at 2000 rpm for 10 minutes and the middle buffy coat was collected after discarding the top plasma layer using a pipette. The huffy coat was mixed with 10 ml of PBS and washed by spinning it at 1500 rpm for 5 minutes. The pellet containing leukocyte and CTCs were resuspended in 600 μl serum free RPMI media for further use.

Culturing of the CTCs in 3-Dimentional (3D) culture media: A 2×-RPMI media was prepared using the powder media; by dissolving it in half the recommended water amount and by adding the recommended sodium bicarbonate. The 2×-RPMI media was filter sterilized and stored at 4° C. till further use. When the buffy coat cells with CTCs were ready, the 3-D media was constituted by mixing 2.5 ml of 1.6% methocel stock, 2.5 ml of 2×-RPMI media and 1000 of human plasma in a 10 ml culture tube. All the contents were mixed slowly by avoiding any bubble formation, and then 100 μl of the buffy coat cells were added and again mixed to it slowly. The contents were then transferred to a 60 mm cell culture dish and incubated for 14 days at 37° C. and in 5% CO₂. The growths of the CTCs were monitored on a daily basis, slowly the leukocytes started dying and the CTCs started forming 3-D colonies. These colonies were then picked up and passaged in similar media conditions and also used for preparing cytopreps or for RNA isolation for further characterization.

Culturing of the CTCs in spheroid forming culture media: Once the buffy coat cells containing the CTCs were ready the media was freshly reconstituted by mixing 0.5 μl human EGF (200 μg/ml), 100 μlof knockout serum and 5 ml of DMEM/F12 working media. The working media contained DMEM/F12 media base with 5 ml non essential amino acid, and 0.5 ml BME (100 μM stock in PBS) and 5 ml of penicillin/streptomycin; it can be premade and stored at 4° C. Similar to the 3-D culturing 100 μl of the buffy coat cell were added to the reconstituted media, mixed and plated on the 60 mm cell culture dish. The growth of the CTCs was monitored for 14 days before they were passaged to fresh media.

Immuno fluorescent staining for characterization of CTCs: The prostate cancer cell lines and cultured CTCs were stained with Cytokeratin (CK), prostate specific antigen (PSA) and CD45 antibodies which are the accepted markers for CTC characterization (CK/PSA+ and CD45). The CK antibody, (sc8018) was from Santa Cruz Biotechnology (Santa Cruz, Calif.) and the PSA (ab9537) and CD45 (ab10559) antibodies were from Abeam (Cambridge, Mass.). The cells and/or CTCs were cyto-preped on the coated glass slides. The cells were then fixed in 4% paraformaldehyde (10 minutes.), permeabilized in 0.1% Triton-x (5 minutes) and then blocked in 10% goat serum (1 hour). They were stained in the primary antibody for 1 hour at RT (CK, 1:25 dilution, PSA 1:50 dilution) or for 16 hours at 4° C. (CD45, 1:200 dilution). Either a FITC- or PE-tagged secondary antibody was used at 1:100 dilutions to detect the markers. The LNCaP and C4-2 cell lines were used as a positive control for CK and PSA staining and melanoma M14 line was used as a negative control. Leukocytes from the healthy volunteer were used as a positive control for CD45 staining and LNCaP and C4-2 were used as negative controls.

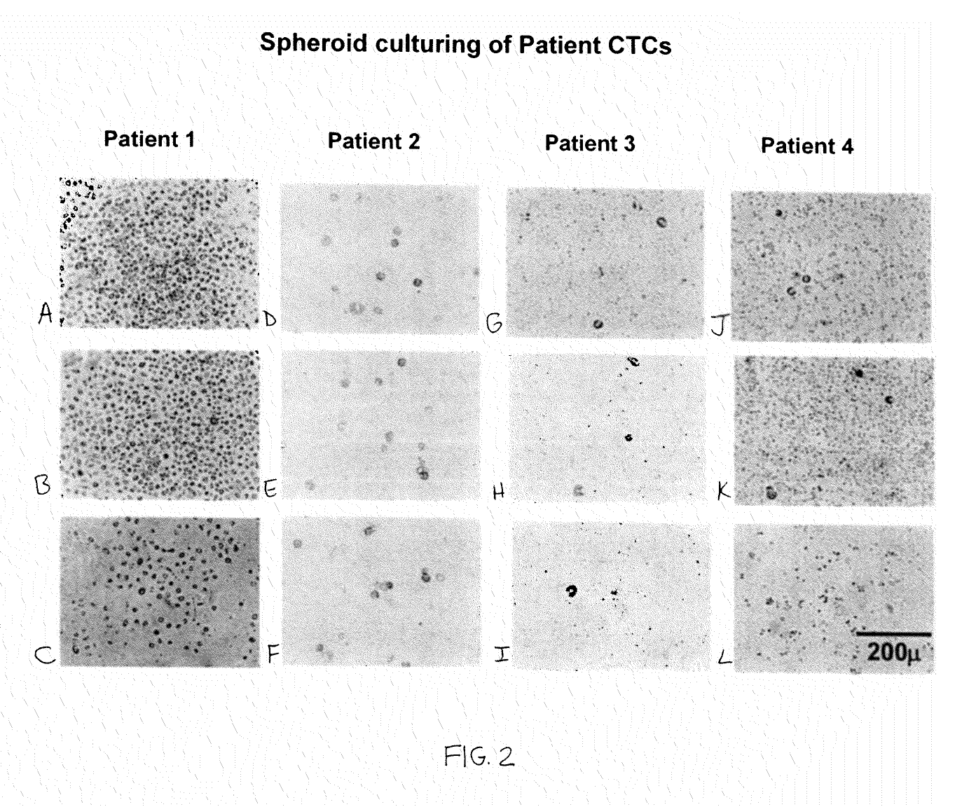

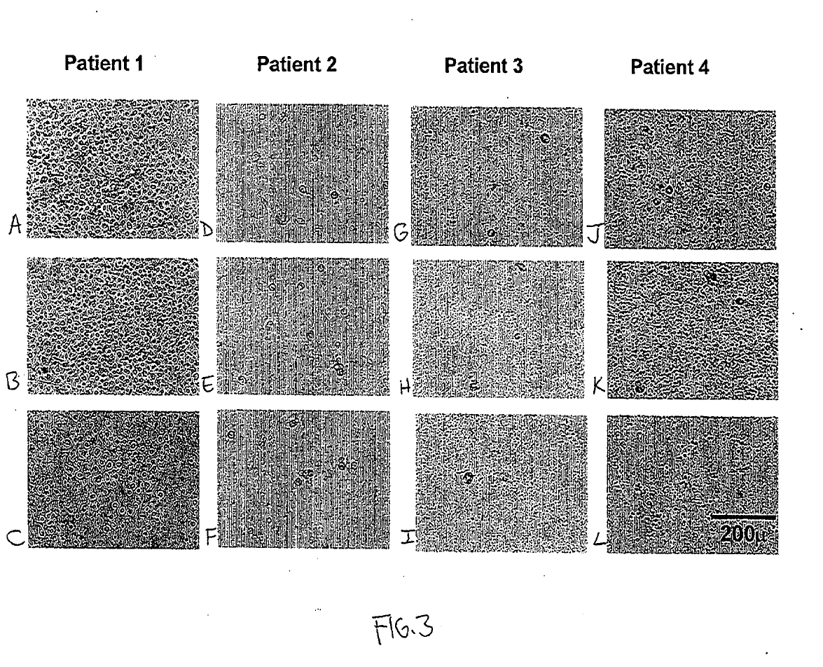

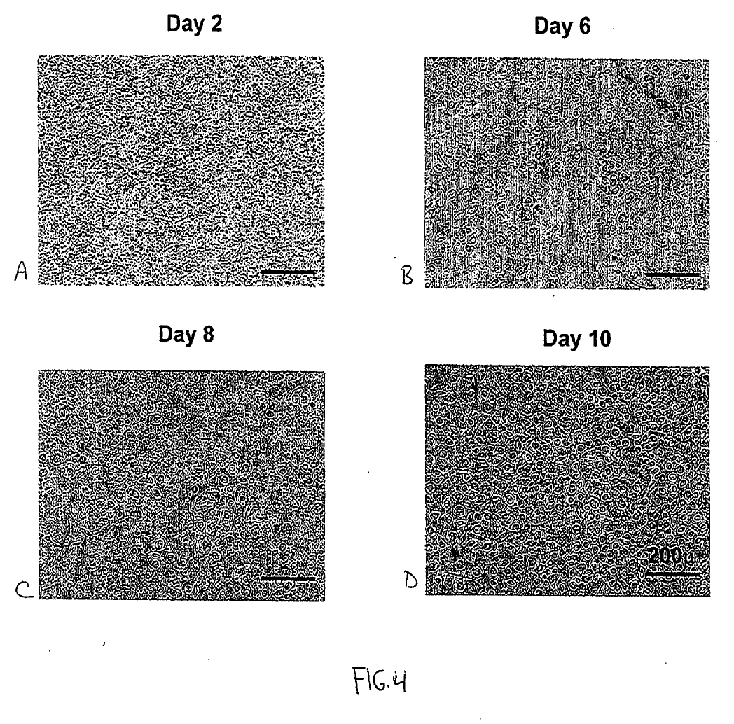

In the spheroid culture media only CTCs from one patient (Patient 1) grew, the other 3 patients (Patient 2, Patient 3, Patient 4) did not have significant growth (FIG. 2). This result may be related to aggressiveness of the cancer or the number of CTCs in the patient sample. The time course of growth of the CTC from Patient 1 in spheroid culture is shown in FIG. 3.

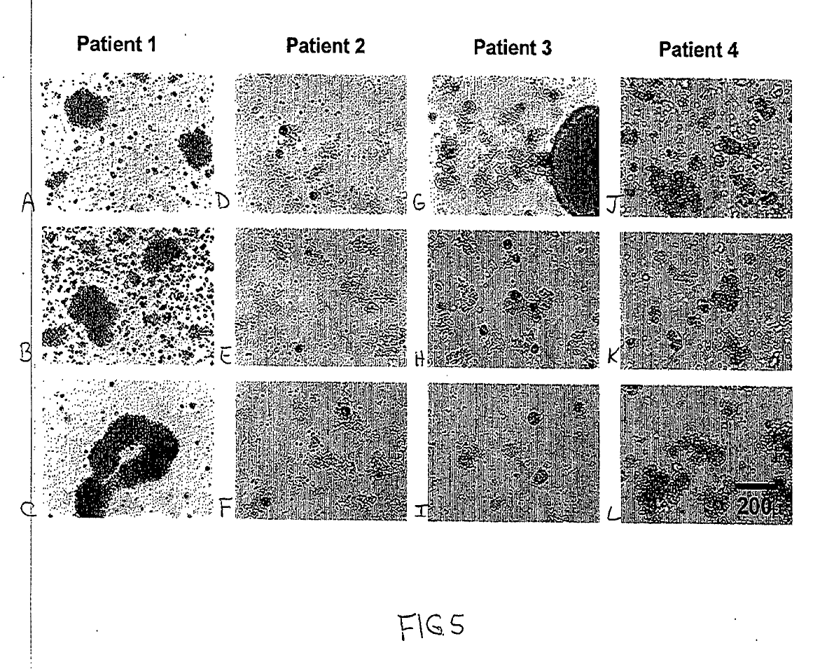

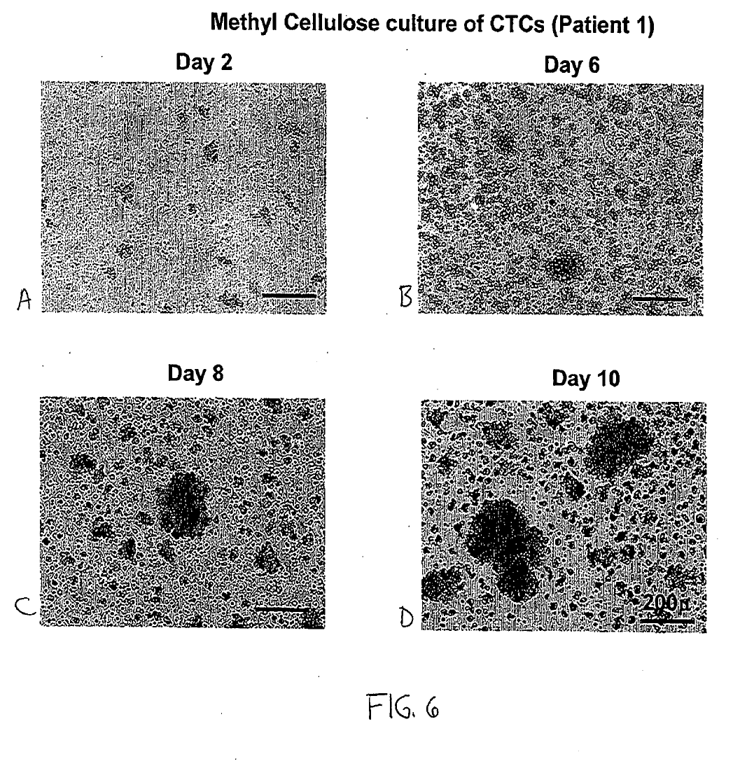

CTCs from all four patients preferentially grew in the three-dimensional culture (FIG. 4). The time course of growth of the CTCs from Patient 1 in the three-dimensional culture is shown in FIG. 5.

The CTCs from the spheroid and three-dimensional cultures were analyzed for CTC markers by immunofluorescence. The CTCs cultured in spheroid media from Patient 1 were analyzed for cytokeratin and CD45 only. The results showed that the cultured CTCs stained positively for cytokeratin, but were negative for CD45. This phenotype is characteristic of CTCs.

The CTCs cultured in the three-dimensional methyl cellulose were analyzed for expression of cytokeratin, PSA, and androgen receptor (AR). The results showed that CTCs expanded from all four patients were universally positive for cytokeratin, and were also positive for PSA. Differential expression of PSA was observed among cells in each sample, and also among patients. CTCs expanded from Patient 1 showed higher PSA expression as compared to CTCs expanded from Patients 2 , 3 and 4. CTCs expanded from Patients 1, 3 and 4 stained positively for AR (CTCs from Patient 2 were not analyzed for AF). Differential expression of AR among cells in each sample, and among patients was noted. Cells from Patient 1 showed higher AR expression as compared to Patients 3 and 4.

### Example 2

### Specific Immunostaining of Melanoma CTC

Isolation of unselected bully coat white blood cells from peripheral blood is performed using Ficoll-Hypaque density gradient or ammonium chloride lysis to deplete erythrocytes and enrich leukocytes.

After washing in patient grade 0.9 Normal Saline, USP, leukocytes are prepared as a thick smear on specially prepared ultraclean glass slides and allowed to settle and adhere. The cells are fixed and permeabilized on the slide with 95% ethanol or 4% paraformaldehyde) and stained. The slides are incubated with an antibody panel that recognize CSPG4 epitopes such as a panel consisting of 5 μg/ml each of the mAb SP10-135, SP10-136, SP10-137, SP10-138, SP10-139 (200 μl/slide) (Soldano Ferrone, University of Pittsburgh) for 30-60 minutes, followed by extensive washes in 0.9N saline. The slides are then stained with Alexa 555-rabit-anti-mouse IgG for 30 minutes, followed by extensive washes in saline. Finally a final wash in double distilled water will be performed. The slides are then counterstained using DAPI to identify nucleated white blood cells and tumor cells from any residual acellular debris.

Slides are then evaluated for melanoma CTC, based on identification of specific fluorescent tumor antigen staining on nucleated cells that do not express leukocyte antigens identified by fluorescent microscopy or computer-assisted digital image analysis technology.

### Example 3

### Detection of Melanoma Cells Based on Intracellular Lipid Content

Buffy coat cells were isolated as described in Example 1, prepared as a thick smear on a glass slide, and stained with a fixed ratio of Nile Red dye (100 nM in Hanks buffered Salt solution) mixed with DAPI (20 nM). This allowed rapid (10 minute) simultaneous staining of melanoma cells derived from human serum based on their lipid content and nuclear DNA for simultaneous verification as nucleated cells. Circulating melanoma cells were specifically identified based on the unique physical property of tumor cells.

Verification of melanoma cell detection was performed using CARS microscopy and Raman micro-spectroscopy. CARS is a four-wave mixing process where two pump laser beams at frequency ωp and a Stokes beam at frequency ωs interact simultaneously with the sample to generate an anti-Stokes signal at the frequency 2 ωp-ωs. The CARS contrast mechanism arises from intrinsic molecular vibration. For example, when the laser frequencies are tuned such that ωp-ωs is equal to 2845 cm-1, which matches the vibrational frequency of CH2 stretch vibration, lipid-rich structures can be directly visualized. CARS microscopy can reduce image acquisition time from many hours in spontaneous Raman microscopy to 20 frames per second with resonance scanning. CARS imaging is label-independent, which allowed for screening of thick smears of subject-derived buffy coat material and identification of potential target cells with fluorescent lipid stains (described above) for efficient second step validation of tumor cells by CARS microscopy.

Raman micro-spectroscopy was also used for lipid composition analysis. CARS microscopy also permits high-speed and chemically selective imaging by focusing all energy into a single vibrational frequency. A typical CARS image lacks the spectral information necessary for the composition analysis of the interested molecules. To overcome this disadvantage, a spectrometer is added to the CARS microscope for spontaneous Raman micro-spectroscopy analysis. The integrated microscope technology allowed for simultaneous high-speed CARS imaging and complete Raman spectra for the composition analysis. White blood cells and tumor cells have differing Raman lipid spectroscopic signatures, further confirming their respective identities.

### Example 4

### Growth of Melanoma CTC in Cell Culture

The buffy coat was isolated from patient peripheral blood using 7.5 ml heparin or EDTA anticoagulated blood specimen, using Ficoll-Hypaque or ammonium chloride lysis. All of the recovered cells (white cells and tumor cells) were plated into methylcellulose (containing 1.6% methyl cellulose, 2×RPMI medium and 2% human serum). The human scrum was derived from outdated fresh frozen serum from the blood bank. The cultures were then grown in a 5% CO2 incubator at 37° C. for 2-4 weeks and tumor cell colonies were evaluated by inverted microscopy. The cells were recovered by micropipette for further characterization, and found to contain cells that express melanoma markers.

1. A method of expanding a circulating tumor cell population, comprising providing a cell population containing circulating tumor cells (CTCs) and culturing the cell population under conditions suitable for preferential expansion of the CTCs.

2. The method of claim 1, wherein cell population containing CTCs is obtained from a subject.

3. The method of claim 1 or 2, wherein said cell population containing CTCs is obtained from a blood sample.

4. The method of claim 3, wherein said blood sample is peripheral blood.

5. The method of claim 1, further comprising obtaining a sample that contains a cell population containing CTC from a subject.

6. The method of claim 5, wherein said sample is a blood sample.

7. The method of claim 6, further comprising substantially removing red blood cells from the blood sample.

8. The method of claim 7, wherein red blood cells are removed using a method selected from the group consisting of fractionation, red blood cell lysis, cell sorting, filtration, adhesion, density centrifugation or combinations thereof.

9. The method of claim 1, wherein the cell population containing CTCs is obtained from a cell culture.

10. The method of any preceding claim, wherein said cell population containing CTCs is cultured in a matrix.

11. The method of claim 10, wherein said matrix is solid or semisolid.

12. The method of any one of claims 1-9, wherein said cell population containing CTCs is cultured in a spheroid culture.

13. The method of claim 10, wherein said matrix is selected from the group consisting of methylcellulose, collagen, and matrigel.

14. The method of claim 13, wherein the depth of said matrix is 0-1 cm.

15. The method of claim 1, wherein said cell population containing CTCs are cultured in media that contains less than about 5% serum.

16. The method of claim 15, wherein said scrum is human serum.

17. The method of claim 1, wherein the phenotype of said CTCs is substantially unchanged during the culturing.

18. The method of claim 1, wherein said cell population containing CTCs are cultured for at least one week.

19. The method of claim 1, wherein the number of CTCs at the end of the culture period is at least about 100× the number of CTCs at the start of the culture period.

20. The method of claim 1, wherein the ratio of CTCs to leukocytes at the end of the culture period is at least about 100:1.

21. The method of claim 1, further comprising incorporation of non-cancer derived extrinsic cells in the culture.

22. A method of detecting circulating tumor cells (CTCs) in a sample, comprising providing a cell sample and detecting lipid-rich structures in the cells contained in the sample, wherein the presence of lipid-rich structure in a cell indicates that the cell is a CTC.

23. The method of claim 22 wherein said detecting is done using a cellular lipid stain.

24. The method of claim 23, wherein said cellular lipid stain is selected from the group consisting of Nile Red, Oil Red O, Dil, DiO, DiA, DiD and DiR.

25. The method of claim 23, wherein said sample is further analyzed using CARS or RAMAN.

26. A method of detecting circulating tumor cells (CTCs) in a sample, comprising staining a cell sample with a reagent that comprises a lipid stain under conditions wherein leukocytes are not stained, but CTCs are stained, and detecting stained cells.

27. The method of claim 26, wherein said reagent comprises a lipid stain at a concentration of 100 nM.

28. The method of claim 27, wherein said reagent further comprises a nuclear stain.

29. The method of claim 28, wherein said nuclear stain is selected from the group consisting of DAPI and Hoeschst 33342.

30. The method of claim 28, wherein said reagent comprises DAPI at 20 nM.
